(19) United States
(12) Patent Application Publication
(10) Pub. No.: US 2014/0031804 A1
(43) Pub. Date: Jan. 30, 2014

(54) DEVICE AND METHOD FOR ABLATING TISSUE

(71) Applicant: Jean-Pierre LALONDE, Candiac (CA)

(72) Inventor: Jean-Pierre LALONDE, Candiac (CA)

(73) Assignee: MEDTRONIC CRYOCATH LP, Toronto (CA)

(21) Appl. No.: 13/559,148

(22) Filed: Jul. 26, 2012

(51) Int. Cl. A61B 18/14; A61B 18/02

(52) U.S. Cl. 606/21; 606/41

(57) ABSTRACT

A method and system for ablating a tissue region. In an exemplary embodiment, the method includes positioning a medical device including a deformable and helical distal portion in contact with a tissue region, the a helical distal portion including a plurality of turns each having a diameter, the diameter of each turn being greater than the diameter of the next distal turn, compressing the helical distal portion against the tissue region, deforming the distal portion into a substantially concentric spiral, and activating one or more treatment elements and ablating the target tissue. The method may further include advancing the uncompressed helical distal portion into a hollow anatomical feature, such as a pulmonary vein, and activating one or more treatment elements and ablating the target tissue.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method and system for creating substantially circular, substantially circumferential, or linear lesions on a tissue region.

### BACKGROUND OF THE INVENTION

Atrial fibrillation (AF) is the most common cardiac arrhythmia, in which disorganized electrical impulses (usually generated by the roots of the pulmonary veins) interrupt the normal electrical impulses generated by the sinoatrial node, which in turn causes an irregular conduction of electrical impulses to the heartbeat-generating ventricles. AF may result from a number of conditions, such as hypertension, coronary artery disease, pericarditis, lunch disease, hyperthyroidism, carbon monoxide poisoning, or rheumatoid arthritis. Indeed, AF itself may increase the likelihood of stroke by as much as sevenfold.

Common methods of treating AF include heart rate control medications (to slow the heartbeat) or rhythm control medicals (to reinstate the normal heartbeat). However, like most medications, these treatments may cause serious undesirable side effects, and constant heart monitoring is often necessary. Other treatment options include synchronized electrical cardioversion or chemical cardioversion, which converts an abnormal heart rhythm to a normal rhythm using electricity or drugs. Catheter ablation is also frequently used, involving a minimally invasive procedure by which areas of cardiac tissue that facilitate the irregular electrical conduction are ablated using any of a number of energy modalities.

If catheter ablation is used, one or more pulmonary veins (PVs) may be targeted. AF is commonly initiated by foci located in the PVs. PVs are large blood vessels that carry oxygenated blood from the lungs to the left atrium (LA) of the heart. In order to disrupt the propagation of abnormal electrical currents, the ablation catheter is placed around the opening of the PV to the heart and/or within the PV where the foci are located. However, the PVs are usually not regularly shaped, and often have an asymmetrical interior that can be difficult to navigate. Further, the openings of two closely positioned PVs may form a single irregular opening, which can make ablation with many currently used ablation elements ineffective (for example, single loop-style ablation elements or the treatment elements of focal catheters). Additionally, the treatment of other types of cardiac arrhythmia may require ablation of tissue in or around the PV and tissue in other areas of the heart. However, it is often necessary to use more than one device in order to effectively destroy aberrant electrical currents. Having to replace a device during surgery can be time consuming, difficult to accomplish, and potentially dangerous for the patient.

Accordingly, an ablation device having one or more ablation elements suitable for treating aberrant electrical currents in cardiac tissue is desired. In particular, the desired device is suitable for treating AF and other arrhythmias by ablating a variety of cardiac tissues, including the pulmonary veins. For example, the device should be capable of ablating foci within the PVs, at the PV opening (ostium), and along the walls of the heart chambers.

### SUMMARY OF THE INVENTION

The present invention advantageously provides a method and system for ablating a tissue region. The method may include positioning a medical device including a deformable and helical distal portion in contact with a tissue region, the a helical distal portion including a plurality of turns each having a diameter, the diameter of each turn being greater than the diameter of the next distal turn, compressing the helical distal portion against the tissue region, deforming the distal portion into a substantially concentric spiral, and activating one or more treatment elements and ablating the target tissue. At least a portion of the substantially concentric spiral may be in contact with a surface of the tissue region. Each of the plurality of turns includes an anterior surface, a posterior surface, and a peripheral surface. A plurality of electrode treatment elements may be arranged on at least one of the anterior surface and the peripheral surface of at least one of the plurality of turns.

The method may further include advancing the uncompressed helical distal portion into a hollow anatomical feature such that at least one surface of at least one turn is substantially in contact with a surface of the anatomical feature, and activating the one or more treatment elements and ablating at least a portion of the anatomical feature. The method may further include placing at least a portion of the peripheral surface of at least one turn in contact with an area of target tissue, and activating the one or more treatment elements and ablating the target tissue in a linear pattern.

The system may include a medical device including a deformable and helical distal portion in contact with a tissue region, the helical distal portion including a plurality of turns, each turn having a greater diameter than the immediately distal turn, and a console including a cryogenic coolant reservoir.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a system including a medical device with a helical distal portion;

FIG. 2A shows a first embodiment of a medical device with a helical distal portion and a shaft;

FIG. 2B shows a second embodiment of a medical device with a helical distal portion and a shaft;

FIG. 3A shows a helical distal portion of a medical device having a plurality of electrodes and compressed or shortened into a substantially concentric spiral;

FIG. 3B shows a helical distal portion of a medical device lengthened into an elongated helix;

FIG. 4 shows a schematic representation of a method of ablating a tissue region;

FIGS. 5A and 5B show a method of compressing the helical distal portion of the medical device to treat a tissue region;

FIGS. 6A and 6B show a second method of shortening the helical distal portion of the medical device to treat a tissue region; and

FIG. 7 shows a method of creating a linear ablation lesion on a tissue region using a device described herein.

### DETAILED DESCRIPTION OF THE INVENTION

As used herein, the term “substantially planar” describes the configuration of the helical distal portion when it is fully contracted or compressed into substantially concentric spiral. Although the substantially concentric spiral is capable of being substantially planar when compressed against a flat surface, for example, it is understood that a tissue region will itself rarely be planar. Therefore, “substantially planar” refers to the fully compressed or shortened distal helical portion (either being substantially in contact with a tissue region or not), whether or nor the tissue region is planar. The term “substantially planar” also refers to the substantially concentric spiral that is created by more than two but fewer than all of the turns of the helical distal portion (for example, when the helical distal portion is partially compressed or shortened).

As used herein, the term “substantially concentric” is used to refer to the spiral that is created when the helical distal portion is compressed or shortened completely or partially. Even if the helical distal portion is partially compressed or shortened, the distalmost two or more turns will be compressed or shortened into a spiral that is substantially planar. As noted above, the term “substantially planar” is used herein with the understanding that a tissue region will itself rarely be planar.

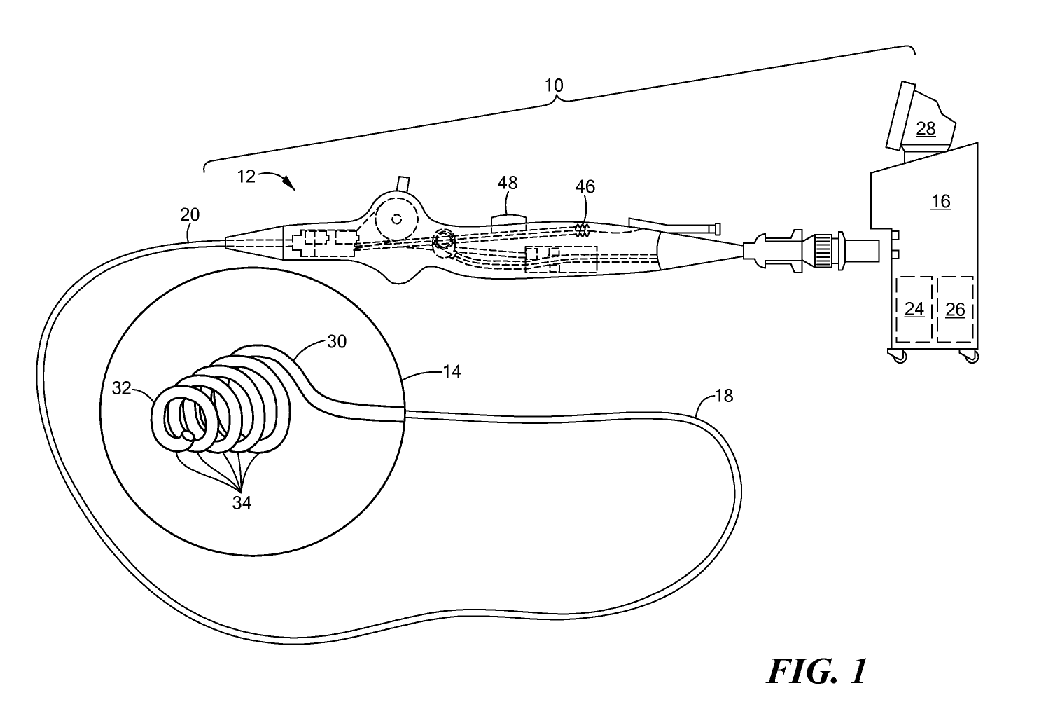

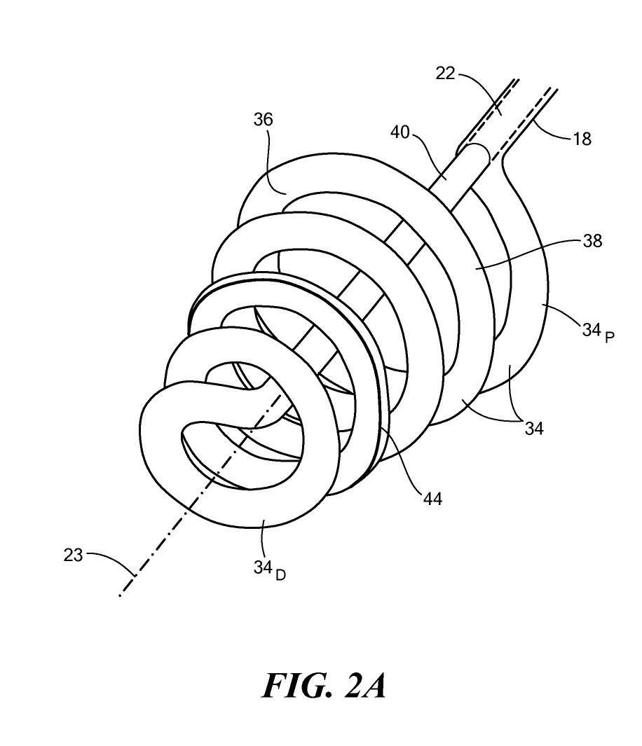

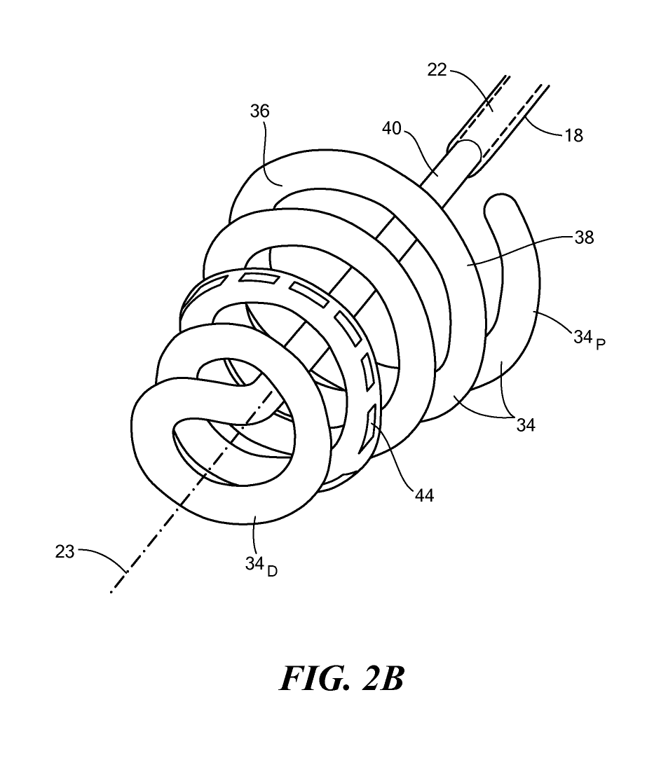

Referring now to FIG. 1, a system 10 including a medical device 12 with a helical distal portion 14 is shown. The system 10 generally includes a medical device 12 for treating a region of tissue 13 (not shown in FIG. 1) and a console 16 for monitoring, adjusting, and controlling various system parameters and device functionality. The medical device 12 includes an elongate body 18 having a proximal portion 20, a helical distal portion 14, and defining one or more lumens 22 therebetween in fluid and/or electrical communication with the console 16. The device 12 further has a longitudinal axis 23 (as shown in FIG. 2A).

Continuing to refer to FIG. 1, the console 16 may be adapted for use with any of a number of energy modalities, including but not limited to, radio frequency (RF) ablation, microwave ablation, ultrasound ablation, cryoablation, and laser ablation. The console 16 may include one or more of a coolant reservoir 24, an energy generator 26 (for example, radio frequency, ultrasound, microwave or laser), a computer with display, and various displays, screens user input controls, keyboards, buttons, valves, conduits, connectors, and power sources for monitoring, adjusting, and controlling system parameters and/or device functionality. The helical distal portion 14 of the device 12 may be in fluid and/or electrical communication with the console 16 via the one or more lumens 22 defined by the elongate body 18 of the device 12.

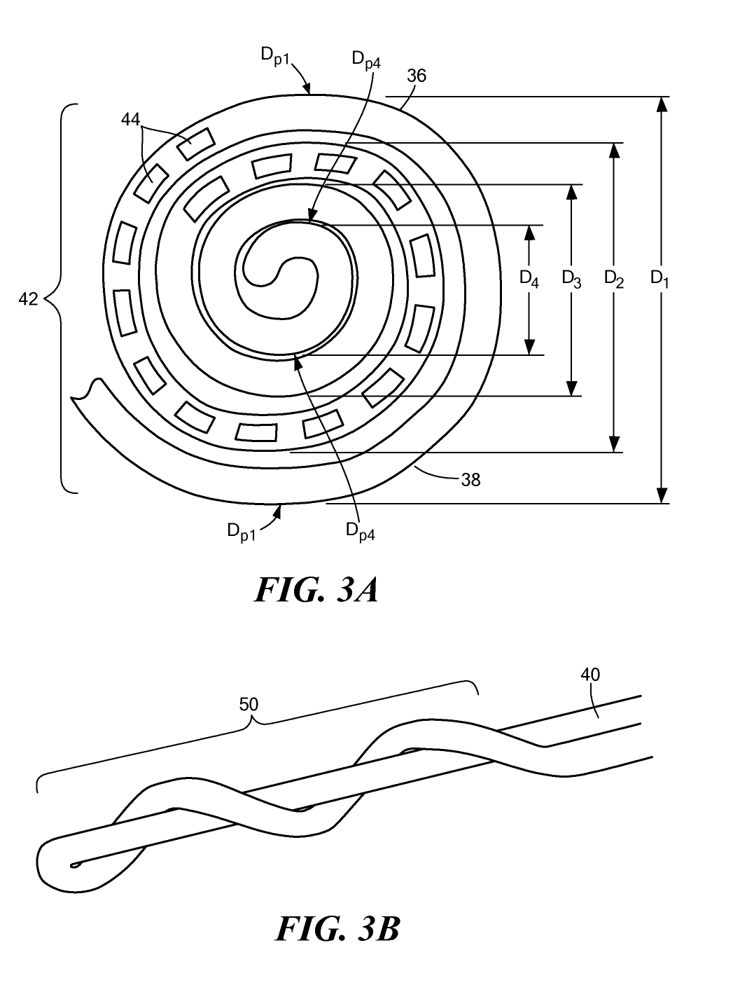

Continuing to refer to FIG. 1, the helical distal portion 14 includes a proximal portion 30, a distal portion 32, and plurality of turns 34. Each turn 34 has an anterior surface 36, a peripheral surface 38, and a diameter Dₙ (as shown and described in FIG. 3A). Further, each turn may be approximately 360 degrees, but may be slightly less or slightly more than 360 degrees. As is shown in the figures, the helical distal portion 14 has a substantially conical shape. Moving in a proximal to distal direction, each turn 34 has a greater diameter than the next turn 34. The device 12 may include a shaft 40 (as shown in FIGS. 2A and 2B) slidably movable within a lumen of the device 12 and coupled to the distalmost turn 34_{D} of the helical distal portion 14, such that longitudinal movement of the shaft 40 will shorten or elongate the helical distal portion 14.

The helical distal portion 14 may be composed of a different material than the elongate body 18 that is flexible and readily deformable. For example, the elongate body 18 may be more rigid than the helical distal portion 14 so that the elongate body 18 may provide support to the helical distal portion 14 when pressure is exerted to compress the helical distal portion 14. Alternatively, the elongate body 18 and helical distal portion 14 may be composed of the same material. Additionally, the helical distal portion 14 may be composed of a thermally or electrically conductive shape memory material (for example, Nitinol) that is biased to a helical geometry. Further, the helical distal portion 14 may instead be an expandable element, such as a cryoballoon. For example, the helical distal portion 14 may be inflated with cryogenic fluid supplied from the console 16 and then used to cryoablate a tissue region 13. Even when inflated with fluid, the cryoballoon may be flexible enough to transition into a substantially concentric spiral 42. Still further, as shown in FIGS. 2 and 3A, the helical distal portion 14 may include a plurality of electrodes 44 or other treatment elements disposed on at least a portion of the anterior 36 and/or peripheral surface 38 of at least one turn 34. In all embodiments, the electrodes 44 or other treatment elements may be in communication with and controllable by the console, such that one or more electrodes 44 or other treatment elements may be activated or deactivated individually, giving the user greater control over the size and shape of the ablation area.

Continuing to refer to FIG. 1, the device 12 may further include a handle 46 with various knobs, levers, user control devices, input ports, outlet ports, connectors, lumens, wires, and the like. The handle 46 may also include one or more actuators 48 that mechanically or electrically shorten or lengthen the helical distal portion 14 (as shown and described in FIG. 2A).

Referring now to FIG. 2A, a first embodiment of a medical device with a helical distal portion and a shaft is shown. As shown and described in FIG. 1, the device 12 may include a shaft 40 disposed and slidably movable within a lumen 22 of the device 12 and coupled to the distalmost turn 34_{D} of the helical distal portion 14, such that longitudinal movement of the shaft 40 will shorten or elongate the helical distal portion 14. Additionally, axial rotation of the shaft 40 relative to the elongate body 18 may increase or decrease the diameter D of the turns 34. The shaft 40 may be coupled to the end of the distalmost turn 34_{D}, with a continuous transition between the helical distal portion 14 of the device 12 and the shaft 40 (as shown in FIG. 2A), or the shaft 40 may be coupled to any other point on the distalmost turn 34_{D} that will allow for shortening of the helical distal portion 14 into a substantially concentric spiral 42 (as shown in FIG. 3A). Longitudinal movement of the shaft 40 may transition the helical distal portion 14 into any configuration between substantially concentric spiral 42 (that is, when the shaft 40 is fully retracted and the distal portion is substantially planar) and configuration wherein the shaft 40 is fully extended and the helical distal portion 14 is spiraled about and proximate the shaft 40 (shown in FIG. 3B) in an elongated helix 50. The proximalmost turn 34_{P} of the helical distal portion 14 may be coupled to or continuous with the elongate body 18. Further, the point of connection between the proximalmost turn 34_{P} and the elongate body 18 should be at a point substantially in the center of the proximalmost turn 34_{P}, such that any forces exerted by the shaft 40 and/or the elongate body 18 on the helical distal portion 14 are distributed evenly throughout the width of the helical distal portion 14.

Referring now to FIG. 2B, a second embodiment of a medical device with a helical distal portion and a shaft is shown. Like the device 12 shown in FIG. 2A, the device 12 of FIG. 2B may include a shaft 40 disposed and slidably movable within a lumen 22 of the device 12 and coupled to the distalmost turn 34_{D} of the helical distal portion 14, such that longitudinal movement of the shaft 40 will shorten or elongate the helical distal portion 14. The shaft 40 may be coupled to the end of the distalmost turn 34_{D}, with a continuous transition between the helical distal portion 14 of the device 12 and the shaft 40 (as shown in FIG. 2A), or the shaft 40 may be coupled to any other point on the distalmost turn 34_{D} that will allow for shortening of the helical distal portion 14 into a substantially concentric spiral 42 (as shown in FIG. 3A). Longitudinal movement of the shaft 40 may transition the helical distal portion 14 into any configuration between substantially concentric spiral 42 (that is, when the shaft 40 is fully retracted and the distal portion is substantially planar) and configuration wherein the shaft 40 is fully extended and the helical distal portion 14 is spiraled about and proximate the shaft 40 (shown in FIG. 3B) in an elongated helix 50. Unlike the device 12 shown in FIG. 2A, the proximalmost turn 34_{P} of the helical distal portion 14 shown in FIG. 2B may not be affixed to or continuous with the elongate body 18. The helical distal portion 14 may be in a substantially concentric spiral 42 when in an initial configuration, and advancing the shaft 40 may decompress the spiral 42 to form the helical 14 configuration.

Referring now to FIG. 3A, a helical distal portion of a medical device having a plurality of electrodes and compressed or shortened into a substantially concentric spiral is shown. When compressed or shortened, the spiral 42 may substantially lie in a plane that is substantially orthogonal to a longitudinal axis 23 of the sheath. As described in FIG. 1, the helical distal portion 14 includes a proximal portion 30, a distal portion 32, and plurality of turns 34. Each turn 34 has an anterior surface 36, a peripheral surface 38, and a diameter Dₙ. The diameter Dₙ of each turn 34 is measured from the widest opposite points Dₚₙ on the peripheral surface 38 of the turn 34 (for example, points Dₚ₁ and Dₚ₄ as shown in FIG. 3A). Although the helical distal portion 14 may include any number of turns 34 (but having at least two), the distal portion 14 of FIG. 3A includes four turns 34. Because of the substantially conical shape of the helical distal portion 14, moving in a proximal to distal direction, each turn 34 has a greater diameter Dₙ than the next turn 34. For example, in FIG. 3A, the diameter of the first turn D₁ is greater than the diameter of the fourth turn D₄.

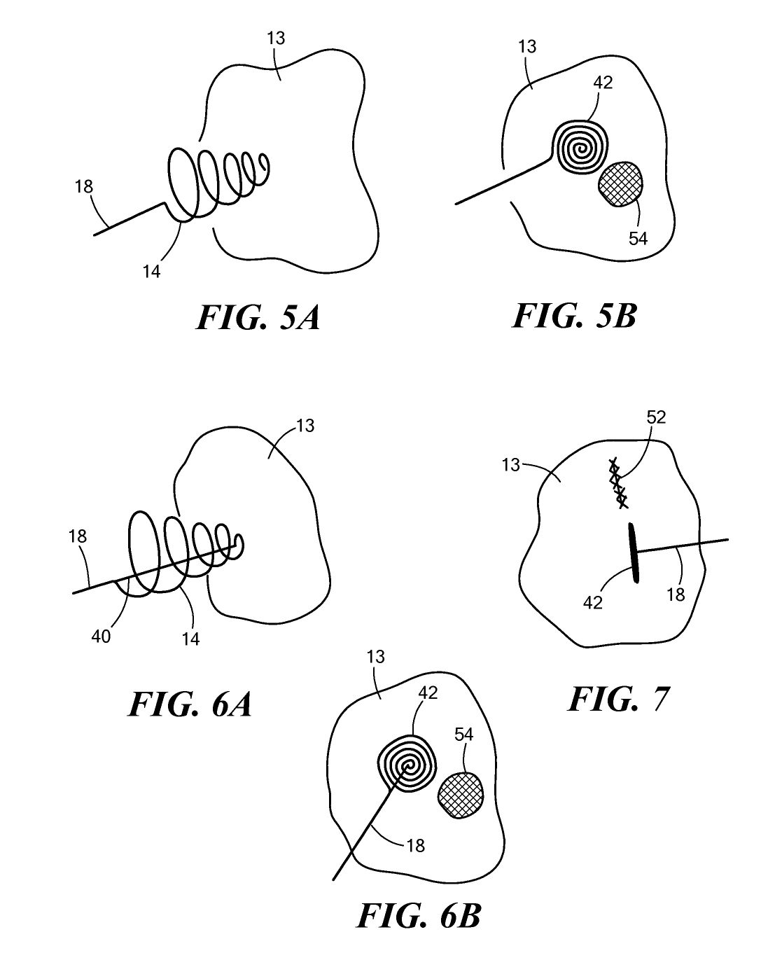

Continuing to refer to FIG. 3A, the helical distal portion 14 may include a plurality of electrodes 44 or other treatment elements disposed on at least a portion of the anterior 36 and/or peripheral surface 38 of at least one turn 34. The electrodes 44 may be band electrodes (as shown in FIG. 2A), discrete electrodes (as shown in FIGS. 2B and 3A), a mesh array of electrodes disposed about at least a portion of the helical distal portion 14, or in any number of configurations, patters, sizes, and shapes. For example, the helical distal portion 14 may be a cryoballoon onto which electrodes 44 are coupled if the system 10 is configured for RF ablation and cryoablation. Electrodes 44 coupled to the peripheral surface 38 of a turn 34 may be used to create a linear lesion 52 on a tissue region 13 (as shown and described in FIG. 7). Similarly, electrodes 44 coupled to the anterior surface 36 of a turn 34 may be used to create a large substantially circular lesion 54 when the helical distal portion 14 is compressed or shortened into a substantially concentric spiral 42 and used to ablate a tissue region 13 (as shown and described in FIGS. 5A-6B). The more turns 34 that include electrodes 44 on the anterior surface 36, the larger the lesion will be when a compressed or shortened helical distal portion 14 is used to treat a tissue region 13.

Referring now to FIG. 3B shows a helical distal portion of a medical device lengthened into an elongated helix. As described above, longitudinal movement of the shaft 40 may transition the helical distal portion 14 into any configuration between substantially concentric spiral 42 (that is, when the shaft 40 is fully retracted and the distal portion 14 is substantially planar) and configuration wherein the shaft 40 is fully extended and the helical distal portion 14 is spiraled about and proximate the shaft 40 (shown in FIG. 3B) in an elongated helix 50. This fully extended configuration has a reduced diameter and may be useful, for example, when advancing the device through an introducer sheath and/or the patient's vasculature or other confined spaces.

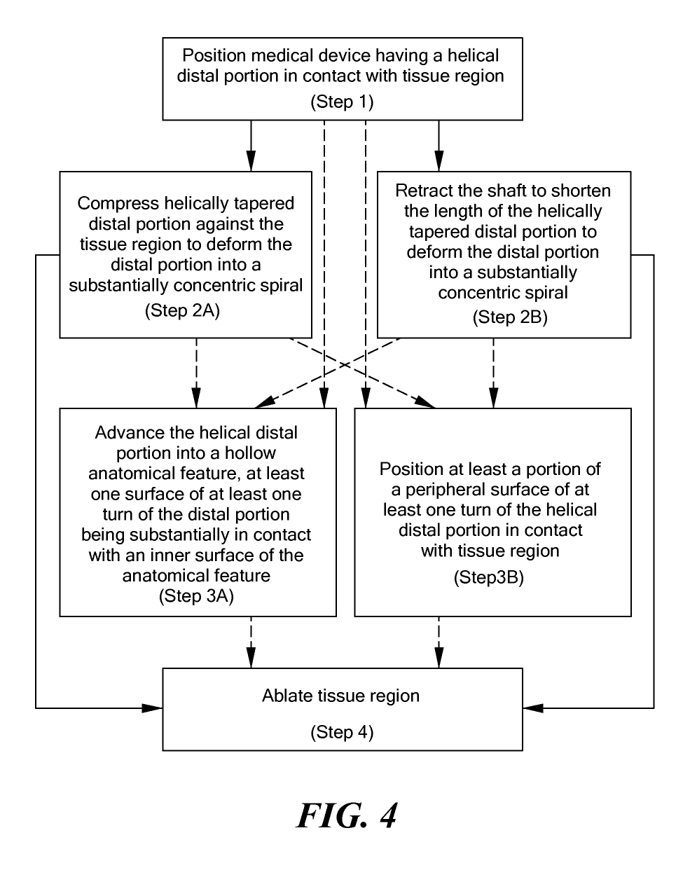

Referring now to FIG. 4, a schematic representation of a method of ablating a tissue region is shown. The method generally includes positioning a medical device 12 having a helical distal portion 14 in contact with a tissue region 13, compressing or retracting the helical distal portion 14 into a substantially concentric spiral 42, and ablating the tissue region 13. In Step 1, the medical device 12 is positioned in contact with a tissue region 13. For example, the medical device 12 may be advanced through the patient's vasculature or placed directly in contact with target tissue 13, for example, cardiac tissue. The tissue region 13 may be continuous, in that there are no significant breaks or gaps in the tissue region 13 (for example, not an area of tissue surrounding a vein opening). The target or desired position of the medical device 12 may be determined through, for example, fluoroscopy or other positioning systems.

In Step 2, the helical distal portion 14 of the device 12 is compressed or shortened into a substantially concentric spiral 42. The helical distal portion 14 may include one or more electrodes 44 or other treatment elements on the anterior surface 36 of at least one turn 34, or the distal portion 14 may be a treatment element (for example, a cryoballoon). When the distal portion 14 is compressed or shortened (and the treatment elements are activated), the anterior surface 36 of at least one turn 34 may then be brought in contact with the tissue 13 to cause ablation. If one or more electrodes 44 on the anterior surface 36 of at least one turn 34 are used, the location of the electrodes 44 may determine the extent to which the distal portion 14 must be compressed or shortened in order to cause ablation by the electrodes 44. If the helical distal portion 14 is as shown in FIG. 1 (without a shaft 40), advancing the device toward the tissue region 13 will mechanically compress the deformable distal portion 14 of the device against the tissue 13, thereby causing the distal portion 14 to transition to a substantially concentric spiral 42 (Step 2A). If the helical distal portion 14 is as shown in FIG. 2A (with a shaft 40), retracting the shaft 40 within the device will cause the distal portion 14 to transition to a substantially concentric spiral 42 (Step 2B). Further, depending on the lesion size desired and the type and location of treatment elements, the distal portion 14 may be partially compressed or shortened so as to bring fewer than all of the turns 34 in contact with the tissue 13.

The method may further include advancing the helical distal portion 14 into a hollow anatomical feature, such as a pulmonary vein, so that at least one surface of at least one turn 34 of the helical distal portion 14 is in contact with an inner surface of the anatomical feature (Step 3A). For example, at least one turn 34 may include one or more electrodes 44 on the peripheral surface 38 and/or the distal portion 14 may be a treatment element (for example, a cryoballoon). When the peripheral surface 38 of the at least one turn 34 comes in contact with the tissue 13 (and the treatment elements are activated), a linear or helical lesion may be created on an inner surface of the anatomical feature (Step 4). Additionally, one or more treatment elements may be deactivated to prevent tissue damage to non-target areas within the PV.

The method may further include positioning at least a portion of a peripheral surface 38 of at least one turn 34 of the helical distal portion 14 in contact with the tissue region 13 to create a linear lesion 52. In this manner, the helical distal portion 14 may be used to create larger, substantially circular lesions 54 (with the anterior surface 36 of at least one turn 34, with the lesion increasing in size with an increase in the number of turns 34 included), may be used to substantially circumferential lesions within a hollow anatomical feature (with the peripheral surface 38 of at least one turn 34), and may be used to create linear lesions 52 (with at least a portion of the peripheral surface 38 of at least one turn 34). To create the linear lesion 52 of Step 3B, the helical distal portion 14 may be oriented such that its longitudinal axis is parallel to a surface of the tissue region 13. In contrast, for example, to create the larger, substantially circular lesion 54 of Steps 2A and 2B, the helical distal portion 14 may be oriented such that its longitudinal axis is perpendicular to a surface of the tissue region 13.

Steps 2A-3B of FIG. 4 are depicted with dashed arrowed lines because, although all of the steps shown in FIG. 4 may be performed, Steps 2A-3B are optional. For example, Steps 1, 4, and either 2A or 2B may be performed to create a substantially circular lesion 54 on a tissue region 13. Optionally, either Step 3A, 3B, or both may also be performed.

Referring now to FIGS. 5A and 5B, a method of compressing the helical distal portion of the medical device to treat a tissue region is shown. Although not shown in FIGS. 5A-7, the device 10 may actually appear as shown in FIGS. 1-3B. Alternatively, the device 10 and/or the helical distal portion 14 may actually appear as in FIGS. 5A-7. For example, the helical distal portion 14 may be a coiled wire suitable for use in RF ablation.

Continuing to refer to FIGS. 5A and 5B, the helical distal portion 14 has a substantially conical shape (as shown and described in FIG. 1). Moving in a proximal to distal direction, each turn 34 has a greater diameter than the next turn 34. Each turn 34 has an anterior surface 36, a peripheral surface 38, and a diameter Dₙ. As is shown and described in FIG. 3A, the helical distal portion 14 may also include a plurality of electrodes 44 at least a portion of the peripheral and/or anterior surface 36 of at least one turn 34. The electrodes 44 coupled to the anterior surface 36 of a turn 34 may be used to create a large substantially circular lesion 54 when the helical distal portion 14 is compressed into a substantially concentric spiral 42 and used to ablate a tissue region 13 (FIG. 5B). The more turns 34 that include electrodes 44 on the anterior surface 36, the larger the lesion will be when a compressed helical distal portion 14 is used to treat a tissue region 13. Alternatively, the helical distal portion 14 may be a treatment element (such as a cooled thermally conductive material or cryoballoon), in which case, the number of turns 34 that are compressed into the substantially concentric spiral 42 will dictate the size of the lesion created. The maximum size of the circular lesion 54 will depend on either the location of the electrodes 44 or the diameter of the largest turn 34 (for example, if the helical distal portion 14 is a treatment element such as a cryoballoon). If electrodes 44 are located on substantially the entire spiral 42 or if the helical distal portion 14 (and therefore spiral 42) is a treatment element, the lesion 54 may be a substantially filled-in circle (as shown in FIGS. 5B and 6B), rather than an annular or circumferential lesion. Further, the turns 34 of the helical distal portion 14 and substantially concentric spiral 42 may be substantially circular (as shown, for example, in FIGS. 5A and 5B), or they may be elliptical, obovoid, irregular, or have another shape. In any configuration, however, the turns 34 are able to substantially nest within each other to create the spiral 42 when the helical distal end 14 is compressed or shortened.

In FIG. 5A, the helical distal portion 14 is positioned proximate the tissue region 13 in an uncompressed configuration. The elongate body 18 is advanced toward the tissue region 13, either manually or by a manual or electrical actuator, to compress the helical distal portion 14 into a substantially concentric spiral 42, as shown in FIG. 5B. The helical distal portion 14 may be completely flattened into a substantially concentric spiral 42, or fewer than all the turns 34 may be included in the substantially concentric spiral 42 (for example, if enough force is exerted from the elongate body 18 to only partially compress the helical distal portion 14). When compressed, the turns 34 may be in contact with each other or they may be spaced a distance apart (as shown in FIGS. 5B and 6B). The substantially concentric spiral 42 can then be used to ablate the tissue region 13.

Referring now to FIGS. 6A and 6B, a method of shortening the helical distal portion of the medical device to treat a tissue region is shown. The method of FIGS. 6A and 6B are substantially similar to those of FIGS. 5A and 5B, except that the helical distal portion 14 is shortened to create a substantially concentric spiral 42 rather than compressed. The helical distal portion 14 of FIGS. 6A and 6B includes a shaft 40 disposed and slidably movable within a lumen of the device (as shown in FIG. 2A) and coupled to the distalmost turn 34_{D} of the helical distal portion 14, such that longitudinal movement of the shaft 40 will shorten or elongate the helical distal portion 14. In FIG. 6A, the helical distal portion 14 is positioned proximate the tissue region 13 in an uncompressed configuration. The shaft 40 is retracted within the device (FIG. 6B), either manually or by a manual or electrical actuator, to shorten the helical distal portion 14 into a substantially concentric spiral 42. The helical distal portion 14 may be completely flattened into a substantially concentric spiral 42, or fewer than all the turns 34 may be included in the substantially concentric spiral 42 (for example, if the shaft 40 is retracted far enough to only partially shorten the helical distal portion 14). The substantially concentric spiral 42 can then be used to ablate the tissue region 13.

Referring now to FIG. 7, a method of creating a linear ablation lesion using a device described herein is shown. As described in FIG. 4, the helical distal portion 14 may be used to create linear lesions 52 with at least a portion of the peripheral surface 38 of at least one turn 34 (a linear lesion 52 is shown in FIG. 7). To create the linear lesion 52 of FIG. 7, the helical distal portion 14 may be oriented such that its longitudinal axis is parallel to a surface of the tissue region 13. The helical distal portion 14 may either be in an uncompressed or unshortened configuration, a fully shortened configuration (as shown in FIG. 7, side view), or some configuration therebetweeen. For example, a helical distal portion 14 without a shaft 40 (as shown in FIG. 1) may be used to create a linear lesion 52 when in an uncompressed configuration, whereas a helical distal portion 14 having a shaft 40 (as shown in FIG. 2A) may be used to create a linear lesion 52 when in either an unshortened or shortened configuration.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of ablating tissue, comprising:
positioning a medical device including a deformable and helical distal portion in contact with a continuous tissue region, the helical distal portion including a plurality of turns each having a diameter, the diameter of each turn being greater than the diameter of the next distal turn;
compressing the helical distal portion against the continuous tissue region, deforming the distal portion into a substantially concentric spiral; and
activating one or more treatment elements and ablating the continuous tissue region.

2. The method of claim 1, wherein at least a portion of the substantially concentric spiral is in contact with a surface of the continuous tissue region.

3. The method of claim 1, wherein the helical distal portion is a treatment element.

4. The method of claim 1, wherein each of the plurality of turns includes an anterior surface, a posterior surface, and a peripheral surface.

5. The method of claim 4, wherein the helical distal portion further includes a plurality of electrode treatment elements arranged on at least one of the anterior surface and the peripheral surface of at least one of the plurality of turns.

6. The method of claim 4, wherein the plurality of electrode treatment elements is arranged on the anterior surface and the peripheral surface of each of the plurality of turns.

7. The method of claim 4, wherein the plurality of electrode treatment elements is arranged on the anterior surface and the peripheral surface of at least one of the plurality of turns.

8. The method of claim 6, wherein the plurality of electrode treatment elements is arranged on the anterior surface and the peripheral surface of each of the plurality of turns.

9. The method of claim 4, further comprising:
advancing the uncompressed helical distal portion into a hollow anatomical feature such that at least one surface of at least one turn is substantially in contact with a surface of the anatomical feature; and
activating the one or more treatment elements and ablating at least a portion of the anatomical feature.

10. The method of claim 9, wherein the hollow anatomical feature is a pulmonary vein.

11. The method of claim 5, further comprising:
advancing the uncompressed helical distal portion into a hollow anatomical feature such that at least one surface of at least one turn is substantially in contact with a surface of the anatomical feature; and
activating at least one of the plurality of electrode treatment elements and ablating at least a portion of the anatomical feature.

12. The method of claim 4, further comprising:
placing at least a portion of the peripheral surface of at least one turn in contact with an area of target tissue; and
activating the one or more treatment elements and ablating the target tissue in a linear pattern.

13. The method of claim 1, wherein the medical device further includes a shaft that is slidably movable within the medical device and affixed to a distal tip of the helical distal portion, longitudinal movement of the shaft changing the diameter of each turn.

14. The method of claim 13, wherein the shaft is a guide wire lumen slidably movable within the medical device, the medical device further including a guide wire slidably movable within the guidewire lumen.

15. A method for ablating tissue, comprising:
placing a medical device having a deformable and helical distal portion in contact with a continuous tissue region;
shortening the length of the helical distal portion and deforming the helical distal portion into a substantially concentric flattened spiral; and
activating one or more treatment elements and ablating the continuous target tissue.

16. The method of claim 15, wherein the medical device further includes a shaft that is slidably movable within the medical device and affixed to a distal tip of the helical distal portion, such that retracting the shaft shortens the length of the helical distal portion.

17. The method of claim 16, wherein the shaft is in communication with an actuator capable of advancing or retracting the shaft within the medical device.

18. The method of claim 16, wherein the helical distal portion includes a plurality of turns, each turn having an anterior surface, a posterior surface, and a peripheral surface, each turn having a diameter that is greater than the diameter of the immediately distal turn.

19. The method of claim 18, wherein the helical distal portion further includes a plurality of electrode treatment elements arranged on at least one of the anterior surface and the peripheral surface of at least one of the plurality of turns.

20. A system for ablating tissue, comprising:
a medical device including a deformable and helical distal portion in contact with a continuous tissue region, the helical distal portion including a plurality of turns, each turn having a greater diameter than the immediately distal turn; and
a console including a cryogenic coolant reservoir.
